(19) United States
(12) Patent Application Publication
(10) Pub. No.: US 2024/0245598 A1
(43) Pub. Date: Jul. 25, 2024

(54) AN EXTRACT OF LINUM USITATISSIMUM SEEDS

(71) Applicant: BASF SE, Ludwigshafen (DE)

(72) Inventors: Sandra Mack; David Graham, Düsseldorf (DE); Markus Weissenegger, Duesseldorf (DE); Juergen Falkowski, Düsseldorf (DE); Antje Zimny, Duesseldorf (DE)

(21) Appl. No.: 18/681,981

(22) PCT Filed: Sep. 9, 2022
(86) PCT No.: PCT/EP2022/075126

§ 371 Date: Feb. 7, 2024

(30) Foreign Application Priority Data
Sep. 14, 2021 (EP) 21196495.2

(51) Int. Cl. A61K 8/9789; A61K 8/34; A61K 36/55; A61Q 5/06; A61Q 17/04

(52) U.S. Cl. CPC A61K 8/9789; A61K 8/34; A61K 36/55; A61Q 5/06; A61Q 17/04; A61K 2236/13; A61K 2236/331; A61K 2236/39; A61K 2236/51; A61K 2236/53

(57) ABSTRACT

The present invention relates to a process for making an extract of Linum usitatissimum seeds. Furthermore, it relates to the extract itself and to a cosmetic composition comprising this extract. Furthermore, it relates to the use of this extract or of the cosmetic composition comprising this extract for hair styling or for conditioning or for protection against UV radiation.

The present invention relates to a process for making an extract of Linum usitatissimum seeds. Furthermore, it relates to the extract itself and to a cosmetic composition comprising this extract. Furthermore, it relates to the use of this extract or of the cosmetic composition comprising this extract for hair styling or for conditioning or for protection against UV radiation.

Seeds of Linum usitatissimum are commercially used to obtain the oil that is contained in these seeds, linseed oil, hereinafter synonymously referred to as linseed oil or linseed fat. This linseed oil is rich in omega-3 fatty acids. Depending on how this oil is obtained, e. g. by pressing or by solvent extraction, the remaining, defatted seeds can be whole defatted seeds, pressed, shredded or pulverized defatted seeds. Hereinafter the defatted seeds, no matter whether they are whole defatted seeds, pressed, shredded or pulverized defatted seeds are referred to as “defatted Linum usitatissimum seeds material”. Depending on how effective the process for removing the oil from the seeds is, the defatted Linum usitatissimum seeds material contains more or less residual fat. At least traces of fat are always present.

Hereinafter flax, common flax and linseed are synonymously used names for the plant Linum usitatissimum.

There is broad field of literature relating to the extraction of material from linseeds for use in a diverse range of applications.

Chen, Shanqiao (2011). China Oils and Fats, 36(11), 58-63 describes, according to an abstract available in English, a process for the ethanolic and subsequent alkaline extraction of lignan from defatted linseed meal in order to prove that the residual products from oil extraction of linseeds can be further utilized albeit on a milligram scale.

WO 2014/174220 A1 describes an acidic hydrolysis of flaxseed mucilage obtained by extracting flaxseeds themselves in a solvent. The acidified solution is then neutralized with a base and performing a series of ultrafiltrations to obtain mixtures of neutral oligosaccharides which can then be used to stimulate the repair process of human skin, improve the strength and flexibility of skin and protect the skin against external factors.

WO 2002/062812 A1 describes a process for extracting the various components contained within cold pressed crushed flaxeseeds, for example fats and fat-soluble compounds and finally secoisolariciresinol diglycoside.

RU 2437552 C1, according to a translation into English, describes a process for extracting edible protein from the non-mucilaginous flaxseed cake for the food industry.

Mueller, Klaus et al., Functional properties and chemical composition of fractionated brown and yellow linseed meal (Linum usitatissium L.), Journal of Food Engineering 98 (2010) p. 453-460 describes a process for preparing an extract of soluble dietary fiber from de-oiled linseed meal in which the de-oiled linseed meal in a first step is extracted in an acid extraction at pH 4.0 at 15° C. and the residual solid is the further extracted in a second step in an alkaline extraction at pH 8.0 at elevated temperature of 35° C. The supernatant of said second step is then precipitated at pH 4.0 at elevated temperature of 35° C. and subjected to centrifugation. The supernatant is then neutralized and spray dried to obtain the soluble dietary fiber. The soluble dietary fiber has the drawback of a rather high protein content of 15.6 to 32.3 wt % and fat content of 0.5 to 0.6 wt % and a rather high viscosity especially at lower temperatures.

In the cosmetics business there is a trend towards more sustainable and environmentally friendly products. Up to now persistent, non-biodegradable synthetic polymers are one of the most important performance giving materials in cosmetic applications, they provide rheology, film-forming, fixation both of hair and of materials to surfaces as well as stabilization of ingredients in heterogeneous systems. While there are current offerings of natural and/or biodegradable alternatives to synthetic polymers, these do not deliver comparable levels of performance to the synthetic materials.

Therefore, the problem underlying the present invention is to provide a bio-based material with advantageous cosmetic properties. Preferably this material shall be biodegradable.

This problem is solved by the extract according the present invention. This extract is obtainable by the process according to the present invention.

The process according to the present invention, which is a first subject of the present invention is a process for making an extract of Linum usitatissimum seeds, comprising the steps of:
a) providing defatted Linum usitatissimum seeds material,
b1) optionally washing this material with ethanol to remove some of the residual fat still contained in the material, or
b2) optionally thermally treating the defatted Linum usitatissimum seeds material at a temperature of 100 to 200° C., preferably 135 to 190° C., preferably 150 to 180° C.,
c) extracting the material from step a) or optionally one of steps b1) or b2) in a first extraction with water at a temperature of 0 to 20° C., preferably 0 to 15° C., preferably 10 to 15° C., and a pH value that is lower than 6, preferably 1 to 5.5, preferably 3 to 5, so that a first liquid and a first solid are obtained,
d) separating the first liquid and the first solid,
e) extracting the first solid obtained in step d) in a second extraction with water at a temperature of 0 to 25° C., preferably 0 to 15° C., preferably 10 to 15° C., and a pH value that is higher than 7, preferably 7.5 to 14, preferably 7.5 to 8.5, so that a second liquid and a second solid are obtained,
f) separating the second liquid and the second solid,
g) heating the second liquid obtained in step f) to a temperature of 80° C. or higher, preferably to a temperature of 80 to 100° C.,
h) cooling the heated second liquid from step g) to a temperature of 60° C. or lower, preferably to 0 to 60° C., preferably 50 to 60° C., so that a precipitate is formed, or
i) removing water from the second liquid obtained in step f), so that a precipitate is formed,
j) removing the precipitate from step h) or i), so that a third liquid is obtained.

The subject-matters of the dependent process-claims are specific embodiments of the present invention.

In the process of the invention the defatted Linum usitatissimum seeds material can be directly used for the first extracting step c).

In an alternative embodiment, the defatted Linum usitatissimum seeds material can optionally be washed with ethanol in optional step b1) to remove some of the residual fat still contained in the material before the first extracting step c).

In a second alternative embodiment, the defatted Linum usitatissimum seeds material can optionally be thermally treated at a temperature of 100 to 200° C., preferably 135 to 190° C., preferably 150 to 180° C. in optional step b2) before the first extracting step c). Said optional thermal treating step is preferably conducted in a dry surrounding so that the optional thermal treating step can also be referred to an optional roasting step. The optional thermal treating step, preferably the optional roasting step, is preferably conducted for 15 min to 10 hours, preferably for 30 min to 7 hours, preferably from 1 hour to 6 hours, preferably from 2 hours to 5 hours.

The optional alterative embodiments of washing with ethanol and thermal treatment both reduce the microbial ballast in the defatted Linum usitatissimum seeds material, whereby the optional thermal treatment turned out to be more effective.

Further the optional thermal treatment reduces the molecular weight Mw of the final extract both in liquid and solid form so that the optional thermal treatment can be used for obtaining a final extract both in liquid and solid form with a predetermined molecular weight Mw. By tuning the molecular weight of the final extract both in liquid and solid form the performance of the final extract both in liquid and solid form can be fine-tuned.

The process according to the present invention is characterized by a first extraction step c) under acidic conditions of a pH value that is lower than 6, preferably 1 to 5.5, preferably 3 to 5 at low temperatures of 0 to 20° C., preferably 0 to 15° C., preferably 10 to 15° C. to obtain a first solid and a second extraction step e) of the first solid obtained from said first extraction step under alkaline conditions of a pH value that is higher than 7, preferably 7.5 to 14, preferably 7.5 to 8.5 at low temperatures of 0 to 25° C., preferably 0 to 15° C., preferably 10 to 15° C. to obtain a second liquid.

Said second liquid is the precipitated either by heating the second liquid to a temperature of 80° C. or higher, preferably to a temperature of 80 to 100° C. in step g) and then cooling said heated second liquid to a temperature of 60° C. or lower, preferably to 0 to 60° C., preferably 50 to 60° C. in step h) or, as an alternative, by removing water from the second liquid in step i), preferably with a distillation column.

Said precipitate is then removed in step j) to obtain the third liquid.

It is thereby preferred that during the steps of transferring the second liquid to the third liquid of process steps g) and h) or, alternatively, process step i) the pH of the liquid is not adapted.

The process of the invention can further comprise process step k):
k) removing of water from the third liquid obtained in step j), so that a third solid is obtained that has a water content of 0-5% by weight, preferably 0-2% by weight, more preferably 0-1% by weight.

Another subject of the present invention is the extract obtainable by the process according to the present invention. This extract can be a liquid (called third liquid in the claims of the present document) or, in a specific embodiment, a solid (called third solid in the claims of the present document).

Further subjects of the present invention are a cosmetic composition (this is the subject-matter or claim 13; claim 14 is a specific embodiment) and the use of the extract according to the present invention or of the cosmetic composition according to the present invention (this is the subject-matter of claims 15 to 17.

The following embodiments are further embodiments of the present invention:
The cosmetic composition according to claim 13 or 14 further comprising at least one polymer for rheology modification selected from the group consisting of xanthan gum, dehydroxoxanthan gum, hydroxypropyl xanthan gum, cellulose gum, algin, diutan gum, tara gum, tragacanth gum, acacia senegal gum extract, acrylamide/sodium acryloyldimethyltaurate copolymer, acrylates/beheneth-25 copolymer, carbomer, guar hydroxypropyltrimonium chloride and xanthan hydroxypropyltrimonium chloride, wherein the at least one polymer for rheology modification is preferably present in an amount of 0.1-4.0% by weight.
The cosmetic composition according to claim 13 or 14 or according to the preceding embodiment further comprising at least one buffering agent chosen from the group consisting of lactic acid, adipic acid, citric acid, ascorbic acid, aminomethyl propanediol, aminomethyl propanol, sodium hydroxide, potassium hydroxide, calcium hydroxide, triethanolamine, diethanolamine, galacturonic acid, glucuronic acid, glutaric acid, monosodium citrate, succinic acid, sodium succinate and sodium citrate, wherein the buffering agent is preferably present in an amount as required to reach a pH value between 4 and 9.
The cosmetic composition according to claim 13 or 14 or according to any of the preceding embodiments wherein the third liquid according to claim 10 or the third solid according to claim 11 is present in an amount of 0.1-5.0% by weight, preferably 0.3-3.0% by weight.
The cosmetic composition according to claim 14 or according to any of the preceding embodiments, which refer to claim 14, wherein the third liquid according to claim 10 or the third solid according to claim 11 is present in an amount of 0.3-3.0% by weight and the at least one surfactant is present in the amount from 0.5-5.0% by weight.

In a specific embodiment of the present invention the extract according to the present invention (in liquid form according to claim 10 or in solid form according to claim 11) comprises 3 to 15, preferably 5 to 12, % by weight protein and 1 to 15, preferably 2 to 10, % by weight starch.

### EXAMPLES

### Example 1: Preparation of an Alkaline Extract (X)

An extract was made by a method comprising the following steps.
1) Providing a dispersion of 0.1 kg the pressed/shredded Linum usitatissimum mass in 1 kg water;
2) Cooling the dispersion to 10-15° C.;
3) Acidifying (until pH 4) the dispersion with acetic acid;
4) Stirring with continuous adjustment of pH and temperature to maintain a constant pH of 4 and a temperature of 15° C. The mixture was stirred for 15 min and had a constant pH of 4 without further need of adjustment;
5) Transferring the dispersion to a centrifuge and centrifuging at 4000 rpm for 10 minutes to separate the solid and liquid phases. The liquid portion (about 0.7 kg) was disposed of and the solid material (about 0.3 kg) was retained;
6) Transferring the isolated (not dried) solid mass into a vessel with stirrer and dispersing it in 1 kg water and cooling it to 15° C.;
7) Adding 50% KOH solution under stirring until a pH of 8 was achieved while maintaining a constant temperature of 15° C.;
8) Stirring the mixture at 15 ºC for 1 hour without any further pH adjustment;
9) Transferring the mixture to a centrifuge and centrifuging it at 4000 rpm for 10 minutes;
10) Decanting the supernatant liquid into a stirred and heated vessel and disposing of the solid layer;
11) Stirring the supernatant at 90° C. for 10 minutes;
12) Cooling the mixture to 55° C. and transferring it to a centrifuge after a precipitate had formed during the heating and stirring;
13) Centrifuging the mixture at 4000 rpm for 10 minutes;
14) Disposing of the gel like solid mass and the retaining the supernatant liquid (0.7 Kg);
15) Transferring 0.7 Kg of the supernatant liquid to a distillation apparatus and concentrating it until a solid content of 5% was determined gravimetrically; this resulted in a stable off-white dispersion;
16) Transferring the dispersion to a freeze drier and drying it to yield 0.01-0.02 kg of an off-white powder (X).

### Example 2: Preparation of an Acidic Extract (Y)

An extract was made by a method comprising the following steps.
1) Providing a dispersion of 0.1 kg the pressed/shredded Linum usitatissimum mass in 1 kg water;
2) Cooling to 10-15° C.;
3) Acidifying (until pH 4) of the swollen/dispersed mass with acetic acid;
4) Stirring with continuous adjustment of pH and temperature to maintain constant pH of 4 and a temperature of 15° C. The mixture was stirred for 15 min and obtained a constant pH of 4 without further need of adjustment;
5) Transferring the dispersion to a centrifuge and centrifuging it at 4000 rpm for 10 minutes to separate the solid and liquid phases. Retaining the liquid portion (0.7 kg) and transferring it to a distillation apparatus;
6) Concentrating 0.7 kg of the liquid to a solids content of 5% in the distillation apparatus; followed by centrifugation at 4000 rpm for 10 minutes to separate the liquid from any precipitate
7) Transferring the remaining liquid to a freeze drier and drying it to an active content of 99.5%; thus 0.02 kg of off-white/yellow powder (Y) was isolated.

### Example 3: Preparation of VOC Formulations

VOC 10 Formulations were prepared according to the following table.

TABLE 1
VOC 10 Formulations

| | Formulation (wt.-%) | | | | | |
|---|---|---|---|---|---|---|
| Ingredients | A | B | C* | D | E | F |
| VP/VA Copolymer | 0.7 | | | | | |
| PVP | | 0.7 | | | | |
| Extract X* | | | 0.7 | | | |
| Extract Y | | | | 0.7 | | |
| Acrylates Copolymer | | | | | 0.7 | |
| Tert-butylacrylamide/ | | | | | | 0.7 |
| Acrylates copolymer | | | | | | |
| Ethanol | 10.0 | 10.0 | 10.0 | 10.0 | 10.0 | 10.0 |
| Parfum | 0.1 | 0.1 | 0.1 | 0.1 | 0.1 | 0.1 |
| Ethylhexyl | 0.05 | 0.05 | 0.05 | 0.05 | 0.05 | 0.05 |
| Methoxycinnamate | | | | | | |
| Panthenol | 0.1 | 0.1 | 0.1 | 0.1 | 0.1 | 0.1 |
| PEG-40 Hydrogenated | 0.5 | 0.5 | 0.5 | 0.5 | 0.5 | 0.5 |
| castor oil | | | | | | |
| Triethyl citrate | 0.05 | 0.05 | 0.05 | 0.05 | 0.05 | 0.05 |
| Aqua | 88.5 | 88.5 | 88.5 | 88.5 | 88.5 | 88.5 |

*formulation according to the present invention

### Example 4: Performance of VOC Formulations in Curl Retention Test

The curl retention of the composition according to the present invention X was tested in standardized VOC 10 pumpspray formulations vs the alternative acidic extract Y and two synthetic polymers, a vinyl pyrrolidone/vinyl acetate (VP/VA) copolymer and a polyvinylpyrrolidone (PVP).

The curl retention of the formulations was measured as follows. Dark brown hair strands of 2 g free hair, 15 cm length held together with a round shaped lace of 1 cm length. The hair strands were bleached with 5% hydrogen peroxide before use. Each polymer was formulated in a solution of ethanol and water with a VOC content of 50% (unless stated otherwise) the pH of the formulations was adjusted to between 6 and 9 using lactic acid or aminomethyl propanol (AMP) (comparative polymer examples without amine or acid functionality do not need to be neutralized in this way, comparative examples with acidic functionality were neutralized with AMP). The hair strands are dipped into de-ionised water for fifteen minutes, excess water was subsequently squeezed out of the strands between two fingers. The wet hair strands were wound onto Teflon curlers with guides to ensure consistent curling over all strands. The curlers with hair strands were then dried at 70 ºC for at least 3 hours and were subsequently cooled overnight. Each hair strand was unwound and attached to a lab stirrer, approximately 2 g of polymer solution was sprayed from a distance of 20 cm from a pump spray device while the hair strand was rotated at 70 rpm. Per polymer sample 5 such strands were prepared. The strands were then allowed to dry for 1 h under ambient conditions on filter paper before being hung on a rack with a scale into a climate chamber at 25° C. and 90% rh the initial length of the strand and the length after 5 h and 24 h was recorded accordingly.

The curl retention (CR) was calculated using the following formula:

$$CR in \% = [ ( L - {L}_{t} ) / ( L - {L}_{0} ) ] \times 100$$

Wherein: L=Length of hair (15 cm)
   L₀=Length of hair curl, start
   Lₜ=Length of hair curl, after a certain period of time (5 h/24 h)

TABLE 2
Curl Retention Results

Curl Retention (%), 90% rh 25° C.

| Formulation | 5 hours | 24 hours |
|---|---|---|
| A | 25.0 +/− 1.0 | 25.0 +/− 1.0 |
| B | 32.4 +/− 2.1 | 24.3 +/− 1.6 |
| C | 78.0 +/− 7.0 | 76.0 +/− 7.0 |
| D | 26.0 +/− 4.0 | 26.0 +/− 4.0 |
| E | 26.7 +/− 3.7 | 25.0 +/− 1.0 |
| F | 26.7 +/− 3.7 | 25.0 +/− 0.0 |

The composition according to the present invention X in formulation C shows a significantly higher performance with curl retention above 70% using only 0.7% of the active compared with the synthetic polymers which gave curl retention in the region of 30% while using the same amount of active matter (0.7%). The acidic extract Y (in formulation D) offered performance on a comparable level with the synthetic polymers.

### Example 5: Preparation of Gel Formulations

Gel formulations with a synthetic rheology modifier were prepared according to the following table

TABLE 3
Gel Formulations

Formulation (wt.-%)

| Ingredients | G | H | I* | J |
|---|---|---|---|---|
| VP/VA Copolymer | 3.0 | | | |
| PVP | | 3.0 | | |
| Extract X* | | | 3.0 | |
| Extract Y | | | | 3.0 |
| Ethanol | 15.0 | 15.0 | 15.0 | 15.0 |
| Parfum | 0.10 | 0.10 | 0.10 | 0.10 |
| PEG-40 Hydrogenated | 0.40 | 0.40 | 0.40 | 0.40 |
| castor oil | | | | |
| Aqua | 80.80 | 80.80 | 80.80 | 80.80 |
| Aminomethyl propanol | 0.20 | 0.20 | 0.20 | 0.20 |
| Carbomer | 0.50 | 0.50 | 0.50 | 0.50 |

*composition according to the present invention

### Example 6: Performance of Gel Formulations in Bending Stiffness Test

The formulations described were tested on hair for bending stiffness according to the following method. Caucasian, dark brown hair strands of 2 g weight and 15 cm length shaped into a rounded form with lace and with a gluing length of 1 cm were used for the test. These hairs were medium bleached with 5% hydrogen peroxide in advance of the testing procedure. 7 such hair strands were used per product to be tested. For the purposes of testing pump-spray type formulations, solutions of the polymer with a 3% polymer content were prepared with a neutralization of 30 wt.-% of available amine functionality using lactic acid. Dilution with ethanol or water was necessary to reach the desired VOC content of the test solution. Each hair strand was dipped into the polymer solution, upon removal from the solution each hair strand was re-formed into the rounded shape and any excess hairs were gathered together by running the strand between the fingers without exerting pressure. This process was repeated once more. The strands were then dipped once again, the excess was then removed by pulling the hair strand (top to bottom) through a specially designed Teflon form, the hair strand was pulled through the Teflon form three times without any further dipping steps. The formed hair strands were then hung vertically in a rack, where the hair strands were left to dry for at least 1 hour under normal laboratory conditions. After drying the rack and strands were placed in a climate chamber at 65% relative humidity and 21° C. overnight. The maximum force needed to break the hair strands was then measured in cN using a device with a 3 point bending stiffness test set-up which can measure force required until breaking point. The process was repeated for the remaining hair strands in order to obtain a mean value of maximum force before breakage. This value was then converted to a percentage by the following equation.

$$\% BS = ( BS / 1000 ) \times 100$$

The results of the bending stiffness test of gel formulations G H, I and J are described in the following table

TABLE 4
Bending Stiffness of Gel Formulations

| | Bending Stiffness | Standard deviation |
|---|---|---|
| Formulation | (cN) | (+/− cN) |
| G | 159 | 12 |
| H | 356 | 30 |
| I | 292 | 17 |
| J | 97 | 13 |

The gels containing 3% synthetic styling polymer (G and H) demonstrated a lower and an upper performance in terms of bending stiffness, the composition according to the present invention X contained in formulation I showed performance approaching the upper end of the scale at the same level of use as a synthetic polymer. The composition Y obtained via the (in formulation J) acidic extraction route showed performance below the lower end of the scale defined by the synthetic examples.

The two previous examples demonstrate that the inventive composition despite being a) a composition based on natural raw materials, and b) biodegradable/non-persistent can compete and even better the performance of synthetic polymers.

### Example 7: UV Protection/Reduction of UV Damage

In order to assess the ability of the product to protect against the damaging effects of UV light the extracts X and Y were applied to flat blond hair strands of 12 cm free hair length, 2 cm width, and with a weight of 0.7 g. 0.5 g of 3% solutions of X and Y were each applied to 10 of the aforementioned hair strands via pipette and distributed using a conventional hair dyeing brush. The hairs were then allowed to dry overnight under controlled temperature (21° C.) and humidity (40% RH) conditions. The amount of damage caused to the hair strands by UV light was quantitatively by determining the resulting temperature of denaturation of the hair proteins (typically 130-150° C. depending on level of damage) via DSC-measurement as described in “Wortmann, Deutz, J. Appl. Polym. Sci. 48 (1993) 137; Wortmann et al., J. Cosmet. Sci. 53 (2002) 219; Istrate et al., Macromol. Biosci. 9 (2009) 805”. The dried hair strands were then exposed to UV light (300-400 nm) at 80 W/m² for 24 h or 48 h always pairwise under controlled temperature (30° C.) and humidity (40% RH) conditions. After exposure the strands are washed with a typical surfactant solution and warm water before drying. Samples of each hair strand were then analysed for protein denaturation temperature via DSC.

The results of the UV protection test are summarized in the following table.

TABLE 5
Denaturation Temperature Untreated vs
Treated Hair after Exposure to UV light

| | Irradiation | Denaturation | Standard Deviation |
|---|---|---|---|
| Hair Sample | Time (h) | Temperature (° C.) | (+/− ° C.) |
| Untreated | 0 | 139.9 | 0.07 |
| Untreated | 24 | 136.2 | 0.07 |
| Untreated | 48 | 133.9 | 0.14 |
| X treated | 24 | 136.8 | 0.09 |
| X treated | 48 | 136.4 | 0.17 |
| Y treated | 24 | 137.2 | 0.05 |
| Y treated | 48 | 134.8 | 0.21 |

The reduction of the denaturation temperature vs that of untreated hair was significantly reduced after exposure to UV light as described for 24 hrs and 48 hrs respectively.

This equates to a >40% improvement in the reduction of UV damage or increased UV protection when using the extract X compared with a placebo, extract Y also offered an improvement vs untreated hair but not as significant as the extract X.

TABLE 6
Percentage Improvement in UV Protection
of Hair vs Untreated Hair

| | (%) Improvement in UV protection vs |
|---|---|
| Sample | untreated hair (48 h irradiation) |
| X | 41.7 |
| Y | 15.0 |

### Example 8: Alkaline Extraction at Elevated Temperature

In comparative example 8 the process of example 1 has been repeated with the difference of maintaining a constant temperature of 50° C. and stirring the mixture at 50 ºC for 1 hour without any further pH adjustment in steps 7) and 8).

Thereby, the alkaline extraction steps at elevated temperature thereby lead to significant hinderance to cleanly separating the performance product from other components due to increased viscosity versus example 1 as can be seen by comparing the separation performance of example 8 with example 1 in the following Tables 7 and 8.

TABLE 7
Comparison of the separation performance
of example 8 and example 1

| | | Example 8: | Example 1: |
|---|---|---|---|
| | | Alkaline extraction | Alkaline extraction |
| Step | Description | 50° C. | 15° C. |
| 9 | Centrifuge | No clear separation, | Clear separation |
| | | therefore, loss of | between liquid and |
| | | product | solid |
| 10 | Decantation | Viscous inhomogeneous | Low viscous solution |
| | | gel like structure, | easy to separate |
| | | difficult to separate | |
| 11 | Concentration | Viscous turbid emulsion, | Low viscous emulsion |
| 12 | Centrifuge | No separation of solids | Clean separation of |
| | | and liquid phase possible | solid and liquid phases |
| | | | possible |

The resultant powder (Z) has been compared to powder (X) obtained from example 1 in behalf of viscosity and spray particle size. Thereby 1% and 2% aqueous solutions and VOC 15 formulations (see Table 1 with 15% ethanol instead of 10% ethanol) were used. The results are shown in Table 8.

TABLE 8
Viscosities and spray particle sizes of formulations containing powder (X) and (Z)

| | | Viscosity | Viscosity | Viscosity | | |
|---|---|---|---|---|---|---|
| | Viscosity | of alkaline | of Alkaline | of Alkaline | Spray | Spray |
| Alkaline | after | extract | extract | extract | particle | particle |
| Extraction | step L | (1% Aqueous, | (2% Aqueous | (1.8% VOC 15 | size d50 | size d90 |
| Temperature | (spindle 3, | spindle 3, | spindle 3, | spindle 3, | (1% | (1% |
| (° C.) | 100 rpm) | 100 rpm) | 100 rpm) | 100 rpm) | Aqueous) | Aqueous) |
| 50° C. (Z) | 640 | 26 | 46 | 79 | 508 | 797 |
| 15° C. (X) | 50 | 19 | 36 | 45 | 211 | 402 |

The warm extracted material has a higher viscosity which leads to a much larger particle when sprayed from a suitable container as a 1% aqueous solution, i.e. the desired fine spray characteristics expected from a cosmetic spray cannot be achieved, the effect is more significant in a typical VOC (Volatile Organic Content) 15 formulation where the volatile component is ethanol, typical for a market tpe pumpspray application.

### Example 9: Roasting of Pressed Linseed Meal Before Extraction

Extracts (X1) to (X5) were made by a method comprising the following steps.
1) Providing a dispersion of 0.1 kg the roasted pressed/shredded Linum usitatissimum mass in 1 kg water;
2) Cooling the dispersion to 10-15° C.;
3) Acidifying (until pH 4) the dispersion with acetic acid;
4) Stirring with continuous adjustment of pH and temperature to maintain a constant pH of 4 and a temperature of 15° C. The mixture was stirred for 15 min and had a constant pH of 4 without further need of adjustment;
5) Transferring the dispersion to a centrifuge and centrifuging at 4000 rpm for 10 minutes to separate the solid and liquid phases. The liquid portion (about 0.7 kg) was disposed of and the solid material (about 0.3 kg) was retained;
6) Transferring the isolated (not dried) solid mass into a vessel with stirrer and dispersing it in 1 kg water and cooling it to 15° C.;
7) Adding 50% KOH solution under stirring until a pH of 8 was achieved while maintaining a constant temperature of 15° C.;
8) Stirring the mixture at 15 ºC for 1 hour without any further pH adjustment;
9) Transferring the mixture to a centrifuge and centrifuging it at 4000 rpm for 10 minutes;
10) Decanting the supernatant liquid and disposing of the solid layer;
11) Transferring 0.7 Kg of the supernatant liquid to a distillation apparatus and concentrating it until a solid content of 5% was determined gravimetrically; this resulted in a stable off-white dispersion;
12) The dispersion was transferred to a centrifuge and centrifuged at 4000 rpm for 10 min
13) The supernatant liquid was transferred to a freeze drier and dried to yield 0.01-0.02 kg of an off-white powder (X).

Thereby, for extracts (X1) to (X5) the roasting procedure was varied by varying the roasting temperature from 150° C. to 180° C. and the roasting time from 1 h to 3 h as shown in Table 9.

TABLE 9
Roasting conditions of extracts (X1) to (X5)

| Sample | Temperature | Time | |
|---|---|---|---|
| X1 | 150° C. | 2 | h |
| X2 | 150° C. | 3 | h |
| X3 | 180° C. | 1 | h |
| X4 | 180° C. | 1.5 | h |
| X5 | 180° C. | 2 | h |

### Example 10: Determination of Microbial Ballast of Extracts

Extract (X1) has been compared with extracts prepared according to the process as described in example 9, for which the pressed linseed meal has been pretreated differently. The aerobic count of the samples indicate the microbial ballast in the sample. The results are shown in Table 10.

TABLE 10
Microbial ballast of extracts

| | Total aerobic count |
|---|---|
| Sample | in extract (CFU/g) |
| Pressed Linseed meal (freshly opened) | >1000 |
| Pressed linseed meal (freshly opened and | <10 |
| roasted at 150° C. for 2 h) | |
| Pressed linseed meal (opened for 7 months and then | <10 |
| roasted at 150° C. for 2 h) | |
| Pressed linseed meal (freshly opened and roasted | 48 |
| at 120° C. for 4 h) | |
| Pressed linseed meal (freshly opened and washed | 800 |
| with ethanol) | |

It can be seen that a pretreatment with a roasting step at 150° C. for 2 h either for freshly opened and roasted pressed linseed meal or for pressed linseed meal which has been opened 7 months before the roasting step both show an exceptionally low aerobic count. A roasting step at 120° C. for 4 h already lowers the aerobic count significantly compared to untreated pressed linseed meal. A pretreatment with ethanol, which is a known method for sterilizing pressed linseed meal, is not as efficient as the roasting pretreatment.

### Example 11: Properties of Pretreated Extracts (X1) to (X5)

The molecular weights Mw of extracts (X1) to (X5) obtained from the process of example 9 were measured. Additionally, the extracts (X1) to (X5) were subjected to the bending stiffness test as described in example 6. The results are shown in Table 11.

TABLE 11
Mw and bending stiffness of extracts (X1) to (X5)

| | | Mw | Bending |
|---|---|---|---|
| Sample | Roasting conditions | (g/mol) | Stiffness (cN) |
| X1 | 150° C. for 2 h | 153000 | 622 |
| X2 | 150° C. for 3 h | 73100 | 627 |
| X3 | 180° C. for 1 h | 63600 | 577 |
| X4 | 180° C. for 1.5 h | 62800 | 435 |
| X5 | 180° C. for 2 h | 56700 | 301 |

It can be seen that the molecular weight of the extracts can be modulated by means of the conditions of the roasting step. The harsher the roasting conditions the lower is the molecular weight.

The molecular weight thereby has a direct influence on the bending stiffness of the extract. The lower the molecular weight the lower is the bending stiffness. Thus, the pretreatment step can be used for fine-tuning the performance of the extracts.

1. A process for making an extract of Linum usitatissimum seeds, comprising:
a) providing defatted Linum usitatissimum seeds material,
b1) optionally washing the material with ethanol to remove some of residual fat still contained in the material, or
b2) optionally thermally treating the defatted Linum usitatissimum seeds material at a temperature of 100 to 200° C.
c) extracting the material from step a) or optionally one of steps b1) or b2) in a first extraction with water at a temperature of 0 to 20° C. and a pH value that is lower than 6, so that a first liquid and a first solid are obtained,
d) separating the first liquid and the first solid,
e) extracting the first solid obtained in step d) in a second extraction with water at a temperature of 0 to 25° C. and a pH value that is higher than 7, so that a second liquid and a second solid are obtained,
f) separating the second liquid and the second solid,
g) heating the second liquid obtained in step f) to a temperature of 80° C. or higher,
h) cooling the heated second liquid from step g) to a temperature of 60° C. or lower, so that a precipitate is formed, or
i) removing water from the second liquid obtained in step f), so that a precipitate is formed,
j) removing the precipitate from step h) or i), so that a third liquid is obtained.

2. The process according to claim 1, further comprising
k) removing water from the third liquid obtained in step j), so that a third solid is obtained that has a water content of 0-5% by weight.

3. The process according to claim 1, wherein the water in the first extraction is acidified with an acid selected from the group consisting of citric acid, lactic acid, acetic acid, formic acid, tartaric acid, and mixtures thereof.

4. The process according to claim 1, wherein the pH value of the water in the second extraction is obtained by adding to a base to the water, wherein the base is selected from the group consisting of KOH, NaOH, aminomethylpropanol (AMP), triethanolamine, and mixtures thereof.

5. The process according to claim 1, wherein the separating of the first liquid and the first solid in step d) is done with a centrifuge.

6. The process according to claim 1, wherein the separating of the second liquid and the second solid in step f) is done with a centrifuge.

7. The process according to claim 1, wherein the removing of water from the second liquid in step i) is done with a distillation column.

8. The process according to claim 2, wherein the removing of water from the third liquid in step k) is done with a freeze dryer or with a spray drier.

9. The process according to claim 1, wherein the amount of water used in the first extraction and in the second extraction is 5 to 20 times the amount of the defatted Linum usitatissimum seeds material provided.

10. A third liquid obtained according to the process of claim 1.

11. A third solid obtained according to the process of claim 2.

12. The third liquid according to claim 10 comprising 3 to 15% by weight protein and 1 to 15% by weight starch.

13. A cosmetic composition comprising the third liquid according to claim 10 and a solvent selected from the group consisting of water, ethanol, isopropanol, n-butanol, n-pentane, and combinations thereof, and optionally further comprising at least one emulsifier, and optionally further comprising at least one pH adjusting agent.

14. The composition according to claim 13 comprising at least one surfactant selected from the group consisting of decyl glucoside, coco-glucoside, polyquaternium-77, disodium coco-glucoside citrate, methyl coco-glucosides, sodium hydroxypropylsulfonate cocoglucoside crosspolymer, ammonium laureth sulfate, sodium cocoamphoacetate, and sodium laureth sulfate.

15.-17. (canceled)

18. A cosmetic composition comprising the third solid according to claim 11 and a solvent selected from the group consisting of water, ethanol, isopropanol, n-butanol, n-pentane, and combinations thereof, and optionally further comprising at least one emulsifier, and optionally further comprising at least one pH adjusting agent.

19. The composition according to claim 18 comprising at least one surfactant selected from the group consisting of decyl glucoside, coco-glucoside, polyquaternium-7, disodium coco-glucoside citrate, methyl coco-glucosides, sodium hydroxypropylsulfonate cocoglucoside crosspolymer, ammonium laureth sulfate, sodium cocoamphoacetate, and sodium laureth sulfate.

20. A method for styling or conditioning hair or skin comprising applying the third liquid according to claim 10 to the hair or skin.

21. A method for styling or conditioning hair or skin comprising applying the third solid according to claim 11 to the hair or skin.

22. A method of protecting hair or skin against UV radiation comprising applying the third liquid according to claim 10 to the skin or hair.

23. A method of protecting hair or skin against UV radiation comprising applying the third solid according to claim 11 to the skin or hair.
